(19) United States
(12) Patent Application Publication
(10) Pub. No.: US 2013/0172788 A1
(43) Pub. Date: Jul. 4, 2013

(54) ELECTRONIC APPARATUS FOR MEASURING THE PATELLAR REFLEX AND OTHER REFLEXES

(71) Applicants: Gerardo Andres Rivas Perlwitz, Santiago (CL); Carlos Humberto Aguilera Arriagada, Santiago (CL)

(72) Inventors: Gerardo Andres Rivas Perlwitz, Santiago (CL); Carlos Humberto Aguilera Arriagada, Santiago (CL)

(21) Appl. No.: 13/807,417

(22) PCT Filed: Jun. 28, 2011
(86) PCT No.: PCT/CL2011/000037

§ 371 Date: Mar. 7, 2013

(30) Foreign Application Priority Data
Jun. 30, 2010 (CL) 707-2010

(51) Int. Cl. A61B 5/11

(52) U.S. Cl. CPC A61B 5/1118
USPC 600/595

(57) ABSTRACT

The invention belongs to the field of electronic instruments for measuring physiological parameters and medical diagnosis.

The patellar reflex is a fundamental part of neurological examination when analysing damage to peripheral neurological structures with central medullary integration.

At present, this reflex is visually assessed by the practitioner. In addition, it would be of interest to the practitioner to obtain information relating to latency and whether the reflex is polykinetic, parameters which are difficult to assess visually. The instruments of the current state of the art are disruptive to the patient, are complicated to position and/or they provide unprocessed information.

The invention enables the reflex to be objectively measured, is minimally disruptive to the patient and calculates the relevant parameters immediately and automatically. It incorporates sensors, electronics, numerical signal processing for the extraction of parameters and elements for delivering the information. The invention is supplemented with the transmission of data to the neurologist via the Internet.

### TECHNICAL FIELD

The present invention relates to the field of electronic instruments applied to the measurement of human physiological parameters and medical diagnosis.

### PRIOR ART

The patellar reflex is a fundamental part of neurological examination when analysing possible damage to peripheral neurological structures (afferent sensory neuron and efferent motor neuron) with central medullary integration.

The reflex travels through afferent sensory fibres, it is integrated at medullar level and sends its efferent motor response via motor fibres which in this case innervate the quadriceps muscle, which contracts suddenly, completing the reflex arc.

The method currently used to assess the patellar reflex is the following. The patient is sitting or lying down with the knee flexed and relaxed. The medical practitioner taps the tendon of the femoral quadriceps immediately below the knee cap with a hammer. This percussion causes the muscle tendon to stretch, responding with a sudden contraction. Depending on the amplitude of the response, the reflexes are measured in degrees on a scale from 0 (null response) to 4 (hyperactive and very brisk).

The development of instruments to measure human movement dates back to the nineteenth century, when the physiologist C. Ludwig invented the kymograph, which is used to measure different types of movement of the human body and not only reflexes. Initially it was purely mechanical and recorded data on paper. In its modern-day versions, these devices incorporate electronics and computers, but are still complex to build and their size prevents them from being portable. The practitioner must interpret the data provided by the graph depending on each situation.

There are instruments in the current state of the art specifically intended for measuring reflexes, but their size makes them invasive, hampering the patient's mobility. A device invented in the United States under U.S. Pat. No. 4,426,099, granted on 13 Mar. 1984, “Instrument for measuring the range of motion associated with a human body joint”, is a device whose operation is based on a gauge for measuring the angle formed between two metal rods. An end of the first rod is fixed to the muscle and an end of the second rod is fixed to the leg. The size of the device is such that the resulting system affects the mobility of a leg, thereby altering the measurements. Another patent that belongs to the field of the present invention is U.S. Pat. No. 6,480,735, “Neuromuscular reflex assessment method”, granted on 12 Nov. 2002. It is an electromyography device that uses a completely different method to that used by the present invention.

There are also earlier academic papers that report the use of MEMS accelerometers to measure the dynamic variables of reflex movements, but these papers have not materialised into commercial devices. A procedure has been disclosed by Mamizuka et al., “Kinematic quantitation of the patellar tendon reflex using a tri-axial accelerometer.” J Biomech 2007; 40(9):2107-11. Epub 2006 Nov 30. An accelerometer is fixed to the ankle of a person. The data are obtained using a signal analyser and a notebook is used for subsequent analysis thereof. A new line of research was also opened by the University of California, Los Angeles, involving the use of MEMS accelerometers fixed to the patient's leg. The objective was to evaluate the utility of these accelerometers to measure the patellar reflex. References include LeMoyne, et al., Biomed. Eng. IDP, UCLA, Los Angeles, Calif., “Evaluation of a wireless three dimensional MEMS accelerometer reflex quantification device using an artificial reflex system” Complex Medical Engineering, 2009. CME. ICME International Conference 9-11 Apr. 2009, pages 1-5 and LeMoyne, et al., Biomed. Eng. IDP, UCLA, Los Angeles, Calif., USA, “Wireless accelerometer reflex quantification system characterizing response and latency”, in: Engineering in Medicine and Biology Society, 2009. EMBC 2009. Annual International Conference of the IEEE, 3-6 Sep. 2009, pages 5283-5286. In South Africa, the paper by Alexander Carlo Busch, “Reflex Sensors for Telemedicine Applications”, Department of Mechanical and Mechatronic Engineering, University of Stellenbosch, South Africa, March 2008, uses integrated sensors to obtain reflex movement data which are analysed using Matlab commercial mathematical software.

With respect to commercial devices, there are devices in the current state of the art based on accelerometers for measuring human movement parameters, such as the LOCOMETRIX device used in gait analysis. There are systems available on the market for measuring the patellar reflex such as Biopac, based on an electric goniometer used to measure knee angle, which requires fixing several sensors to the patient's skin.

None of these developments, patents or lines of research have given rise to portable devices capable of measuring and processing the signals in real time, due to which doctors do not have devices that are easy to use and can be objectively interpreted to measure the patellar reflex.

### DISCLOSURE OF THE INVENTION

### Technical Problem that it Resolves

The current problem is that the response of the patellar reflex or Achilles reflex or that of other reflexes is subjectively assessed by the practitioner. In the case of high values obtained on the reflex scale, this assessment has satisfactory precision, but in the case of low values, it is difficult for the professional to assess the intensity of the reflex response.

Additionally, it would be of interest to the professional to learn about other reflex characteristics such as latency and to determine whether or not the reflex is polykinetic, which are extremely difficult to assess visually.

The instruments of the current state of the art provided to the professional are disruptive to the patient, must be fixed using various fixations to several parts of the patient's limbs and/or the information provided to the practitioner must be interpreted after performing the measurement, and this interpretation is made based on unprocessed data.

### Operating Principle of the Invention

The present invention allows a completely objective measurement of the variables of the reflex response, with minimum disruption to the patient, and is capable of producing immediate results, automatically calculating the relevant parameters.

The components of the invention include electronics for obtaining data from the movement of sensors, algorithms for extracting information from said data and the elements for delivering the relevant information to the practitioner by means of visual or auditory elements, in addition to elements for transferring the recorded data to computers or Internet sites.

The acceleration, speed and/or movement of the leg are measured using small electronic sensors, which are less than ten centimetres in size along their maximum dimension and have a maximum weight of tens of grammes. There is also the possibility of using a sensor that measures the inclination or a goniometer, maintaining the basic principle of the method of the present invention.

The dynamic variables measured by the sensors are mathematically processed to calculate the latency and amplitude and to determine whether the reflex is polykinetic. This processing can be performed in the same device that houses the sensors or can be performed in a device that is in a different box but that together with the first device comprises a single portable and indivisible unit, connected wirelessly or by cables.

The characteristic reflex variables that have been measured or calculated based on the data are displayed to the practitioner, recorded in the memory or transferred to a computer to keep patient records, and can even be analysed using system identification algorithms to obtain additional information.

### BRIEF DESCRIPTION OF THE DRAWINGS

FIG. 1 shows the most adequate position for fixing to the end of the device that contains the sensor(s) (1).

FIG. 2 shows a conceptual diagram of the main components of the invention. The element (2) is a sensor or several sensors that measure dynamic variables. A numerical analogue converter (3) takes samples of the sensor signals at a predefined frequency, i.e. greater than double the bandwidth of the sensor signal. The numerical data are processed by a microcontroller (4). If necessary, the microcontroller (4) will store the data in a buffer memory (5) and/or transmit them to another device by means of a transmitter (6). The microcontroller processes the data numerically and the result is converted into a signal that outputs a light display (7) to indicate patellar reflex intensity.

FIG. 3 shows the device (8) mounted on a fixation element (9), for fixing the device (8) to the extremity, in such a manner that the light display (10) is clearly visible to the practitioner.

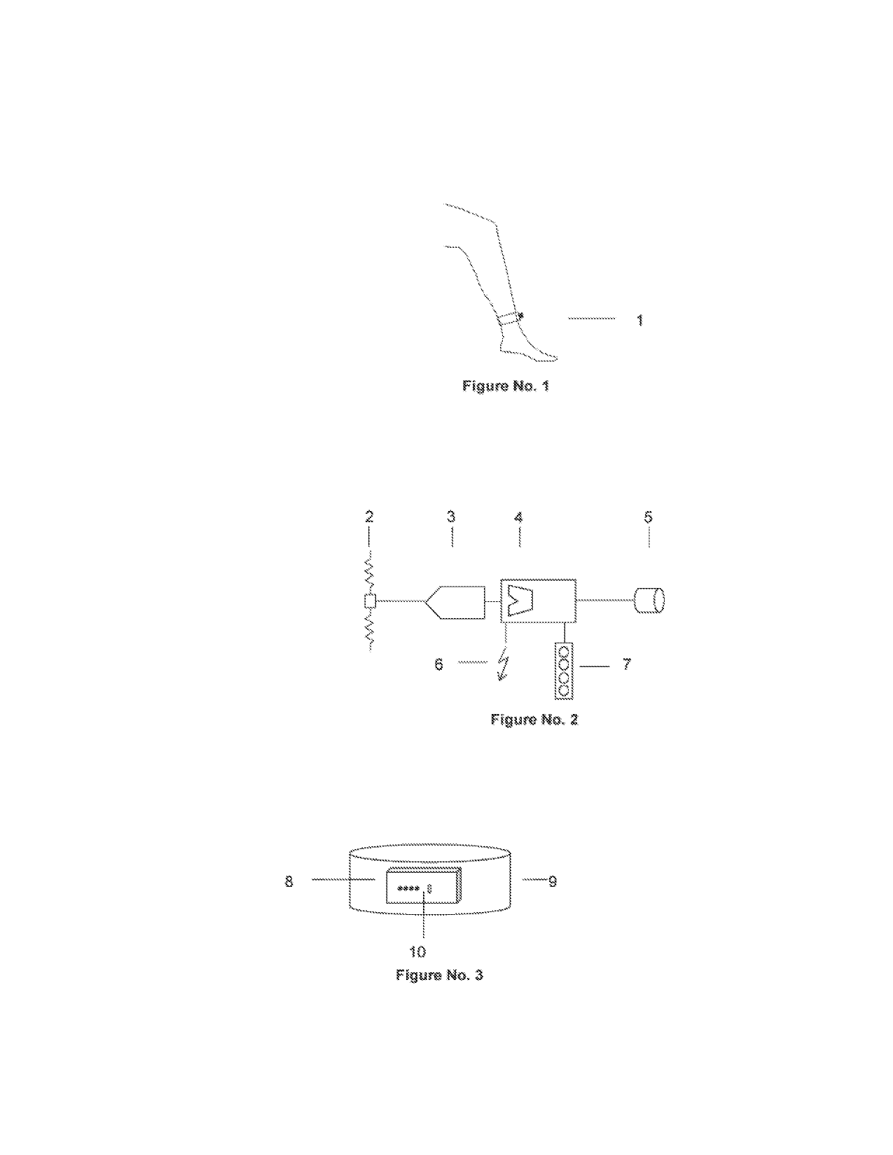

### PREFERRED EMBODIMENTS OF THE INVENTION

In all the embodiments of the invention, a device containing at least one sensor is fixed to the patient's ankle or leg to measure limb movement variables. In this description, the sensor used is an integrated MEMS accelerometer.

When the leg moves, the accelerometer records the acceleration. Given that the movement is pendular, i.e. the knee remains fixed in its position while the leg moves, this angular acceleration translates into a linear tangential acceleration equal to ωR and a radial acceleration equal to ω²R, where ω is the instant rotation speed and R is the distance between the knee and the place where the sensor is fixed. The sensor records the linear and radial accelerations, together with the gravitational acceleration which, for this embodiment, is an alteration that must be considered during mathematical processing.

In one embodiment of the invention, a numerical analogue converter is used to sample the accelerometer's output signal at a sampling frequency in accordance with the Nyquist frequency. There are also integrated accelerometers that measure acceleration and analogue-to-digital conversion in the same block or chip, producing a numerical output signal.

In both cases, a microcontroller is used both to obtain the sensor data and to perform full numerical processing, due to which everything is performed in a single device that is solidarily connected to the leg. The microcontroller can calculate, based on the variables measured by the sensor, the amplitude of the reflex on a scale of 0 to 4 and display the calculated value on an alphanumeric screen. The device can also be embodied in such a manner as to include external optoelectronic lights or diodes which, according to the scale value, turn on the respective light. The microcontroller can also perform digital filter processing to eliminate noise or eliminate the drift constants using, for example, a Kalman filter.

A second embodiment of the invention uses two devices. The first of these, which is very light weight and small in size, is fixed to the leg. It contains the sensors and performs numerical processing to indicate the reflex scale. Simultaneously, it sends data to a second external device that processes them numerically to calculate other parameters such as latency or to determine if it is a polykinetic reflex. This second device is larger in size due to the fact that it can include an alphanumeric screen to deliver all the data to the practitioner. This second device is also portable and can incorporate the function of keeping a historical record of each patient.

The data can be transmitted between the device fixed to the patient's leg and the external device using both wireless means and cables. In all cases there is the possibility of recording, storing and transferring the unprocessed sensor data for subsequent processing for research purposes.

The characteristics of the frequency spectrum of the stimulus applied by the hammer enable different types of numerical processing, including wavelets and autoregressive models for identifying parameters and modelling the response. Therefore, it is possible to reduce sensor drift, reduce noise and obtain a parameter vector space for full reflex identification.

Another embodiment of the invention involves the use of devices containing the described elements installed in-factory.

This is the case of some cell telephone models that incorporate a MEMS accelerometer, a microcontroller and short-range wireless transmission modules such as Bluetooth or data transmission via cell telephone networks.

### INDUSTRIAL APPLICATION

The proposed use of the invention is in the manner of an instrument for the early detection of possible damage to peripheral neurological structures with central medullary integration. This detection can be performed at home by the patients themselves, provided they have an instrument that measures the reflex objectively. This is of special interest to diabetic patients or patients under treatment with immunosuppressants. For commercial application, software that enables the data to be downloaded from the device object of the present invention to a computer is considered necessary. The data can be sent via the Internet to a medical specialist or delivered on optical media or a flash memory.

### ORIGINALITY OF THE INVENTION

The originality of the invention consists of the fact that a portable, compact system is achieved with the capacity to process the data automatically to objectively indicate the relevant parameters of the patellar reflex in real time. A kymograph is a large device that is not specifically designed to measure the patellar reflex and does not automatically provide the reflex parameters. The device patented in the United States under U.S. Pat. No. 4,436,099 is based on a goniometer and must be fixed simultaneously to the thigh and the leg. The size of this device affects leg mobility, due to which the measurements are altered. It is not designed to provide the practitioner with an objective measurement quickly and automatically. The device patented in the United States under U.S. Pat. No. 6,480,735 uses the non-invasive electromyography method, which is completely different from that used by the present invention.

The academic papers by Mamizuka et al., LeMoyne et al. and Alexander Carlo Busch disclose systems that combine sensory elements and commercial computer programs for data analysis. Therefore, they are neither commercial systems nor are they designed to deliver an objective measurement to the professional quickly and automatically.

Commercial products such as LOCOMETRIX are used to obtain gait test data. The Biopac system is based on an electric goniometer for measuring knee angle and requires fixing several sensors to the patients skin, due to which it is disruptive. Neither does it provide the practitioner with all the relevant information.

1-15. (canceled)

16. An electronic device for measuring at least a patient's body reflex, comprising:
at least one sensor for measuring at least one variable relating the movement of a patient's limb, and
a processing element for mathematically processing the variable(s) received from the sensor(s) in order to determine at least one parameter of the reflex.

17. The device of claim 16, wherein the sensor(s) comprise an accelerometer.

18. The device of claim 16, wherein the sensor(s) comprise an inclinometer.

19. The device of claim 17, wherein the sensor(s) further comprise an inclinometer.

20. The device of claim 16, further comprising a fixation element for fixing the sensor(s) to the limb.

21. The device of claim 20, wherein the processing element is configured so as to determine whether the fixation element is correctly fixed to the limb.

22. The device of claim 16, further comprising a displaying element for displaying the at least one parameter in a audio and/or visual way.

23. The device of claim 16, wherein the parameters comprise the amplitude of the reflex, the displaying elements comprising optoelectronic lights or diodes for displaying the amplitude of the reflex according to a predetermined scale.

24. The device of claim 16, further comprising a buffer memory for storing the variable(s) and/or the parameter(s).

25. The device of claim 24, wherein the buffer memory is configured so as to store the variable(s) and/or the parameter(s) in historical patient's records.

26. The device of claim 16, further comprising a transmitter for transmitting to an external device or to an Internet site the variable(s) and/or the parameter(s).

27. The device of claim 16, wherein the processing element further comprises a digital filter for eliminating noise or eliminating drift constants.

28. The device of claim 16, wherein the sensor(s), the fixation element and the processing element are integrated in one single first block.

29. The device of claim 16, wherein the first block comprises a cell telephone incorporating a MEMS accelerometer, a microcontroller and short-range wireless transmission modules.

30. The device of claim 23, comprising:
a single second block integrating the sensor(s) and the fixation element;
a first processing element comprised in the second block for determining the amplitude of the reflex;
the displaying means, comprised in the second block for displaying the amplitude of the reflex; and
a third block, wherein the parameters comprise the latency and the polykinetic nature of the reflex, the third block comprising second processing means for processing the variable(s) received from the sensor(s) in order to determine the latency and the polikinetic nature of the reflex,
the second processing means being connectable to the second block by means of a wireless connection or by cables.

31. The device of claim 30, wherein the third block further comprises an alphanumerical screen for displaying the parameters of the reflex.
